(19) United States
(12) Patent Application Publication
(10) Pub. No.: US 2025/0170287 A1
(43) Pub. Date: May 29, 2025

(54) UV STERILIZER FOR A VEHICLE

(71) Applicants: HYUNDAI MOTOR COMPANY, Seoul (KR); KIA CORPORATION, Seoul (KR); SL CORPORATION, Daegu (KR)

(72) Inventors: Jae Seung Lee, Hwaseong-si (KR); Hyun Woo Choi, Daegu (KR); Kwang Soo Park, Daegu (KR); Myeong Joo Seo, Gyeongsan-si (KR); Ji Hoon Yang, Gyeongsan-si (KR)

(73) Assignees: HYUNDAI MOTOR COMPANY, Seoul (KR); KIA CORPORATION, Seoul (KR); SL CORPORATION, Daegu (KR)

(21) Appl. No.: 18/601,672

(22) Filed: Mar. 11, 2024

(30) Foreign Application Priority Data
Nov. 23, 2023 (KR) 10-2023-0164019

(51) Int. Cl. A61L 2/10; A61L 2/24

(52) U.S. Cl. CPC A61L 2/10; A61L 2/24; A61L 2202/11; A61L 2202/25

(57) ABSTRACT

An ultraviolet (UV) sterilizer for a vehicle includes a main body part mounted in the vehicle. The main body part has a storage area formed therein. A sterilization part is stored in the storage area and can be deployed from the storage area to selectively emit ultraviolet rays to a vehicle interior. A driving part is coupled to the main body part and configured to provide rotational driving force to allow the sterilization part to be selectively withdrawn into and deployed from the storage area. A controller is configured to selectively control operations of the main body part, the sterilization part, and the driving part.

### CROSS-REFERENCE TO RELATED APPLICATION

This application claims, under 35 U.S.C. § 119(a), the benefit of and priority to Korean Patent Application No. 10-2023-0164019, filed on Nov. 23, 2023, the entire contents of which are incorporated herein by reference.

### BACKGROUND

### (a) Technical Field

The present disclosure relates to an ultraviolet (UV) sterilizer for a vehicle. More particularly, the present disclosure relates to a UV sterilizer for a vehicle that can selectively switch from a storage state to an operation state for sterilization and to expand a sterilization area in the vehicle.

### (b) Background Art

In general, plasma emitted to perform sterilization is known to be harmless, even when emitted to the human body. Here, ultraviolet rays (for example, germicidal UV or ultraviolet-C or UVC) have better sterilization power than that of plasma, but it is known that ultraviolet rays are harmful when emitted to the human body.

In addition, although ultraviolet rays have various effects such as sterilization, disinfection, deodorization, and oxidative decomposition of non-biodegradable organic substances, ultraviolet rays are harmful when emitted directly to the human body. Accordingly, if ultraviolet rays are carefully used not to be directly exposed to the human body, the same have an excellent effect of managing household items.

Further, in consideration of various infectious pathogens (for example, COVID-19) in the surrounding environment, people often use various means such as, for example, a portable disinfection kit or the like, to disinfect their hands.

Particularly, after entering a vehicle, a driver normally disinfects his or her hands by exposing the hands to a portable disinfection kit before touching various objects such as a steering wheel and a center console.

However, it may be difficult to reliably perform sterilization only through driver's disinfection. Accordingly, in order for a driver to be safe from infectious pathogens in a vehicle interior, it is necessary to primarily disinfect the vehicle interior.

Therefore, ultraviolet LEDs are installed in the vehicle to disinfect the entire vehicle interior. It is important to ensure safety due to exposure to ultraviolet LEDs and to install ultraviolet LEDs in the vehicle without negatively impacting or deteriorating interior design of the vehicle.

### SUMMARY OF THE DISCLOSURE

The present disclosure has been made in an effort to solve the above-described problems associated with the prior art. It is an object of the present disclosure to provide an ultraviolet (UV) sterilizer for a vehicle configured to use UV rays to sterilize the vehicle interior and to selectively switch from a storage state to an operation state for sterilization. A safety problem and negative interior design effects of the vehicle are thereby prevented due to the UV sterilizer installed in an exposed state. Further, the UV sterilizer has a plurality of ultraviolet-C (UVC) light emitting diodes (LEDs) mounted on a mounting guide having inclinations corresponding to each other and having a reflector mounted on the UVC LED. A sterilization area in the vehicle is thereby expanded when the UV sterilizer switches to the operation state for sterilization.

In one aspect, the present disclosure provides a UV sterilizer for a vehicle. The UV sterilizer includes a main body part mounted in the vehicle. The main body part has a storage area formed therein. The UV sterilizer also includes a sterilization part stored in the storage area and configured to be withdrawn from the storage area to selectively emit ultraviolet rays to a vehicle interior. The UV sterilizer also includes a driving part coupled to the main body part and configured to provide rotational driving force to allow the sterilization part to be deployed from the storage area. The UV sterilizer also includes a controller configured to selectively control operations of the main body part, the sterilization part, and the driving part.

In an embodiment, the main body part may include a cover member formed or configured to shield an open interior of the main body part in the vehicle interior. The sterilization part may be mounted on the cover member.

In another embodiment, the cover member may include a U-shaped connection member connected to the driving part through a rotation shaft. The connection member is configured to rotate to allow the sterilization part to be selectively deployed from or withdrawn into the storage area.

In still another embodiment, the sterilization part may include a mounting frame mounted on the cover member and a heat sink coupled to the mounting frame and configured to be cooled by an air flow generated by driving of a cooling fan. The sterilization part may also include a plurality of UVC LEDs mounted on the mounting frame in a state of facing the heat sink and formed to emit the ultraviolet rays at a plurality of locations.

In yet another embodiment, the sterilization part further may include a reflector mounted on each UVC LED and formed to expand an emission range of the ultraviolet rays.

In still yet another embodiment, the UVC LEDs may be disposed at intervals on a first surface and a second surface of the mounting guide. The first surface and the second surface may respectively have inclinations corresponding to each other with respect to a central portion of the mounting guide.

In a further embodiment, the driving part may include: a housing coupled to a rear side of the main body part; a driving motor coupled to an inside of the housing and configured to provide the rotational driving force; a gear member connected to the driving motor and configured to transmit the rotational driving force generated from the driving motor in response to an input control signal; a clutch gear engaged with the gear member to be rotated in conjunction with the gear member; a shaft member connected to a connection member provided in the sterilization part through a rotation shaft; and a clutch plate locked in a locking groove provided in the shaft member to be rotated in conjunction with the shaft member.

In another further embodiment, the clutch plate may be disposed to make surface contact with an inner peripheral surface of the clutch gear and may be rotated in conjunction with the clutch gear rotated by the rotational driving force transmitted from the gear member, thereby rotating the shaft member.

In still another further embodiment, the clutch plate may be rotated in conjunction with rotation of the shaft member when the sterilization part is manually stored in the storage area and may generate rotational friction in a state of making surface contact with an inner peripheral surface of the clutch gear.

In yet another further embodiment, the driving part may further include an elastic member mounted on an outer peripheral surface of the shaft member and configured to allow a stopper member to fix a position thereof. The elastic member may provide elastic force to the clutch plate to allow the clutch plate to be fixed in the locking groove.

In still yet another further embodiment, the controller may be configured to provide the rotational driving force to the driving part to deploy the sterilization part and open the storage area and to control, upon determining that the storage area is open, the operation of the sterilization part for emission of the ultraviolet rays.

In a still further embodiment, the controller may be configured to provide, upon determining that a predetermined sterilization time has elapsed, reverse rotational driving force to the driving part so that the sterilization part is rotated and is withdrawn into and stored in the storage area.

Other aspects and embodiments of the disclosure are discussed herein.

It should be understood that the terms “vehicle”, “vehicular”, and other similar terms as used herein are inclusive of motor vehicles in general. Such motor vehicles may encompass passenger automobiles including sport utility vehicles (SUV), buses, trucks, various commercial vehicles, watercraft including a variety of boats and ships, aircraft, and the like. Such motor vehicles may also include hybrid vehicles, electric vehicles, plug-in hybrid electric vehicles, hydrogen-powered vehicles, and other alternative fuel vehicles (e.g., fuels derived from resources other than petroleum). As referred to herein, a hybrid vehicle is a vehicle that has two or more sources of power, such as for example, a vehicle powered by both gasoline and electricity.

The above and other features of the disclosure are discussed herein.

### BRIEF DESCRIPTION OF THE DRAWINGS

The above and other features of the present disclosure are described in detail with reference to certain example embodiments thereof illustrated in the accompanying drawings, which are given hereinbelow by way of illustration only, and thus are not limitative of the present disclosure, and wherein:

FIG. 1 is a diagram showing an ultraviolet (UV) sterilizer for a vehicle according to an embodiment of the present disclosure;

FIGS. 2A, 2B, 3A, and 3B are diagrams showing a structure of an UV sterilizer for a vehicle according to an embodiment of the present disclosure;

FIGS. 4 and 5 are diagrams showing an operation state of an UV sterilizer for a vehicle according to an embodiment of the present disclosure;

FIGS. 6 and 7 are diagrams showing an installation state and an operation state of an UV sterilizer for a vehicle according to an embodiment of the present disclosure;

FIGS. 8A-8D are diagrams of an embodiment showing a transmission path of a rotational driving force for an UV sterilizer for a vehicle according to an embodiment of the present disclosure;

FIG. 9 is a diagram showing an embodiment of a structure of a driving part of an UV sterilizer for a vehicle according to an embodiment of the present disclosure and shows a rotational driving force transmission sequence thereof;

FIGS. 10A and 10B are diagrams of another embodiment showing a transmission path of a rotational driving force for an UV sterilizer for a vehicle according to an embodiment of the present disclosure; and

FIG. 11 is a diagram of another embodiment showing a structure of a driving part of an UV sterilizer for a vehicle according to an embodiment of the present disclosure and shows a rotational driving force transmission sequence thereof.

It should be understood that the appended drawings are not necessarily drawn to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

In the figures, the same reference numbers refer to the same or equivalent parts of the present disclosure throughout the several figures of the drawings.

### DETAILED DESCRIPTION

Hereinafter, reference is made in detail to various embodiments of the present disclosure, examples of which are illustrated in the accompanying drawings and described below.

Advantages and features of the present disclosure and a method of achieving the same should become more apparent with reference to the embodiments described below in detail and the accompanying drawings.

While the technical concept of the disclosure is described in conjunction with various embodiments, it should be understood that the present description is not intended to limit the disclosure to the disclosed embodiments. On the contrary, the disclosure is intended to cover, not only the embodiments disclosed herein, but also various alternatives, modifications, equivalents, and other embodiments, which may be included within the spirit and scope of the disclosure as defined by the appended claims.

In describing the embodiments disclosed herein, where it was determined that a detailed description of publicly known techniques to which the disclosure pertains may have obscured the gist of the present disclosure, detailed descriptions thereof have been omitted. When a controller, component, device, element, part, unit, module, or the like of the present disclosure is described as having a purpose or performing an operation, function, or the like, the controller, component, device, element, part, unit, or module should be considered herein as being “configured to” meet that purpose or perform that operation or function. Each controller, component, device, element, part, unit, module, and the like may separately embody or be included with a processor and a memory, such as a non-transitory computer-readable media, as part thereof.

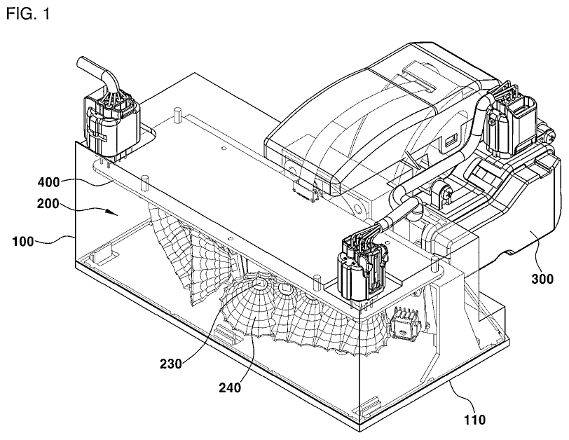

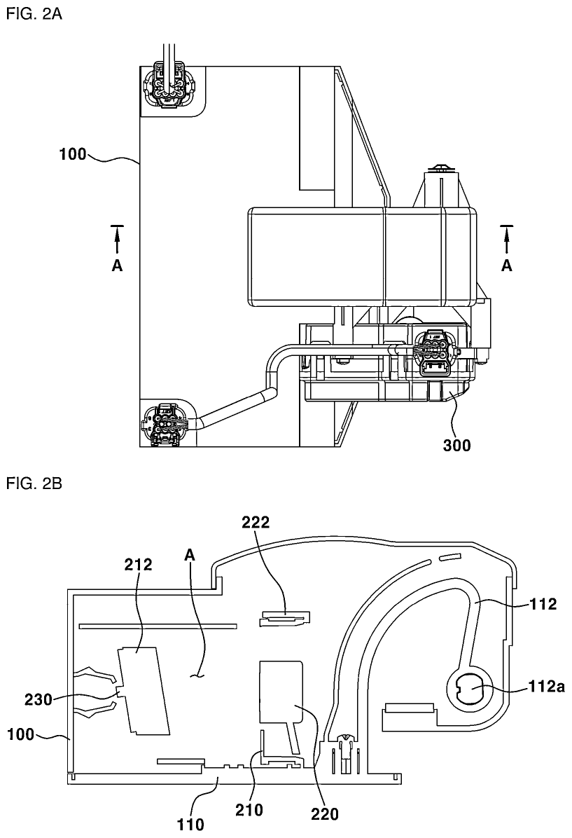

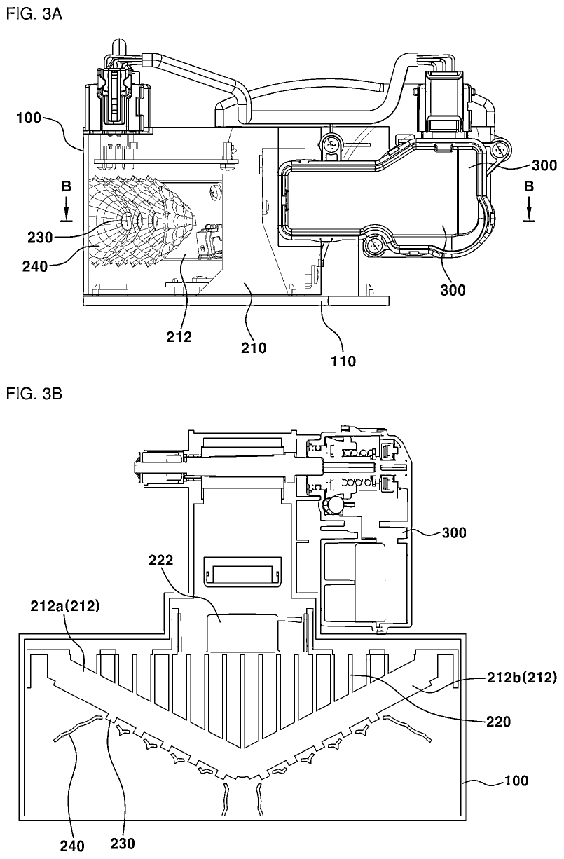

FIG. 1 is a diagram showing an ultraviolet (UV) sterilizer for a vehicle according to an embodiment of the present disclosure. FIGS. 2A, 2B, 3A, and 3B are diagrams showing a structure of an UV sterilizer for a vehicle according to an embodiment of the present disclosure.

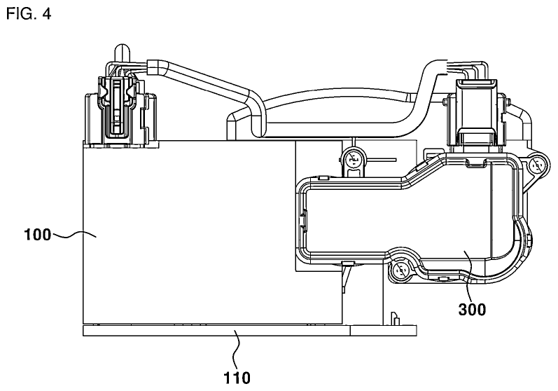

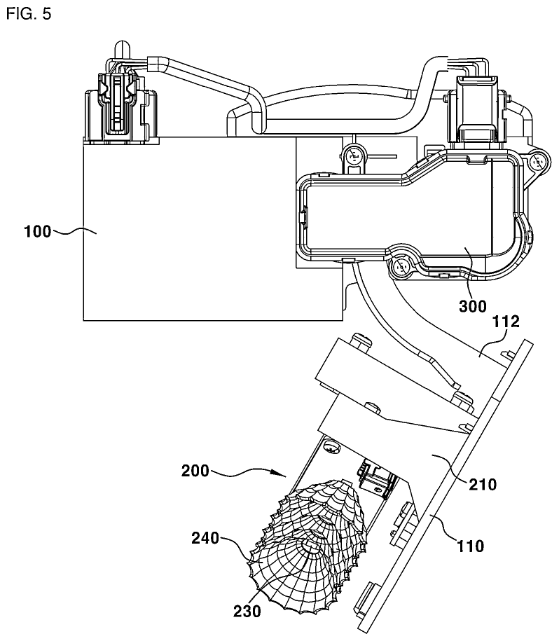

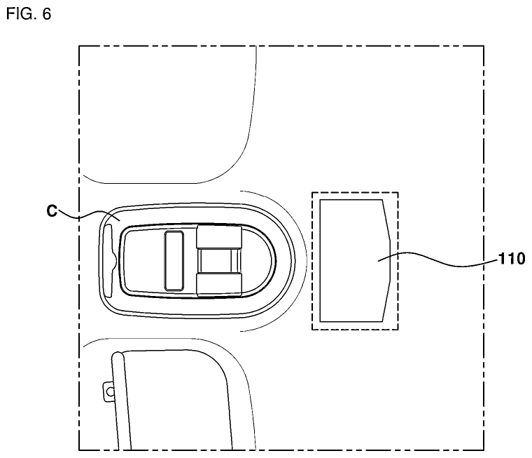

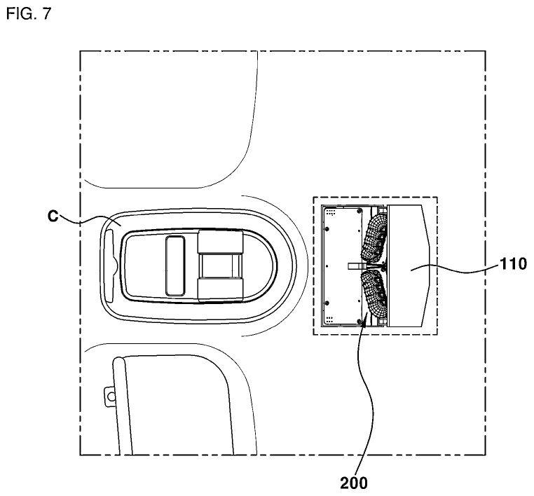

FIGS. 4 and 5 are diagrams showing an operation state of an UV sterilizer for a vehicle according to an embodiment of the present disclosure. FIGS. 6 and 7 are diagrams showing an installation state and an operation state of an UV sterilizer for a vehicle according to an embodiment of the present disclosure and within a vehicle interior.

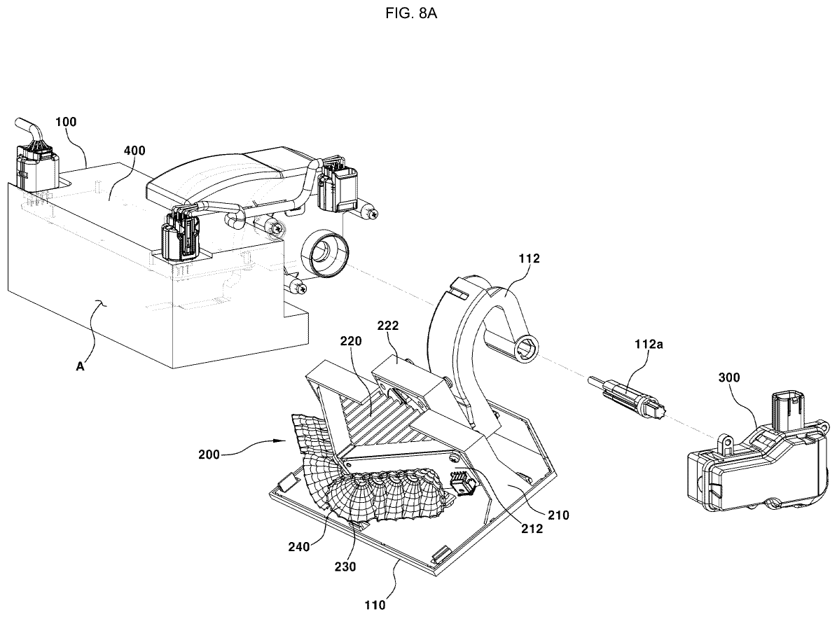

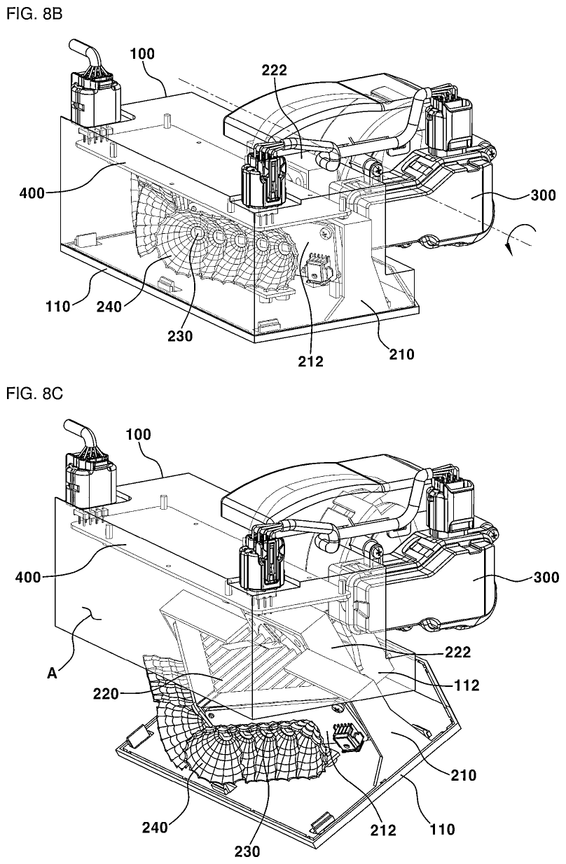

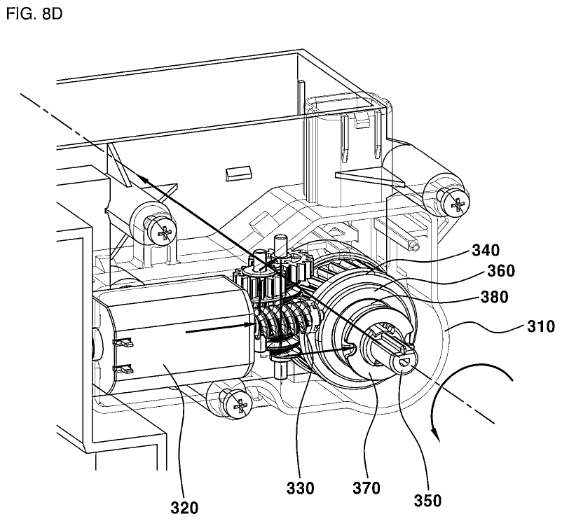

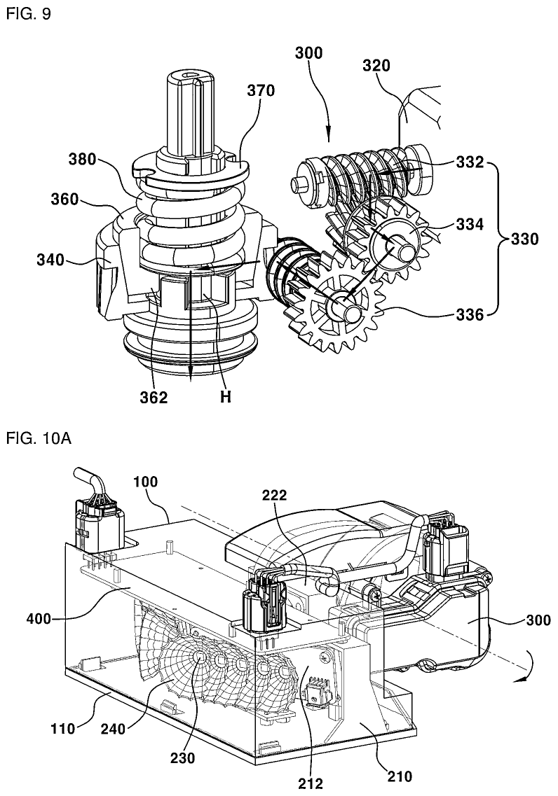

FIGS. 8A-8D are diagrams of an embodiment showing a transmission path of a rotational driving force of an UV sterilizer for a vehicle according to an embodiment of the present disclosure. FIG. 9 is a diagram showing an embodiment of a structure of a driving part of an UV sterilizer for a vehicle according to an embodiment of the present disclosure and shows a rotational driving force transmission sequence thereof.

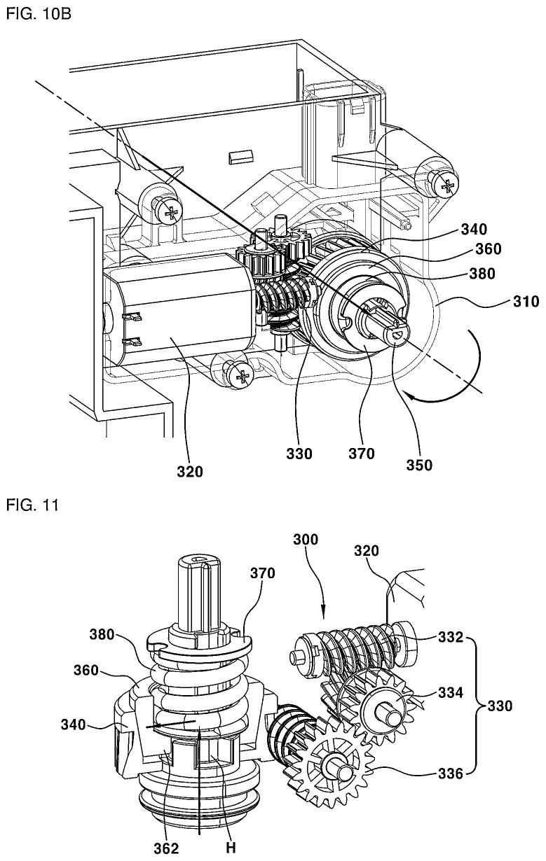

FIGS. 10A and 10B are diagrams of another embodiment showing a transmission path of a rotational driving force of an UV sterilizer for a vehicle according to an embodiment of the present disclosure. FIG. 11 is a diagram of another embodiment showing a structure of a driving part of an UV sterilizer for a vehicle according to an embodiment of the present disclosure and shows a rotational driving force transmission sequence thereof.

Generally, ultraviolet or UV rays are electromagnetic waves having shorter wavelengths than those of visible light and longer wavelengths than those of X-rays. Further, ultraviolet rays have a range of wavelengths form 10 nm to 400 nm and are referred to as UV rays.

In the case of sterilization using UV rays, scientific verification has been conducted on a specific range of wavelength having a sterilizing effect. However, the specific range of wavelength has the shortest wavelength and the highest energy in UV rays, so it is very harmful to human health.

In other words, a wavelength around 265 nm is most effective in sterilizing viruses and bacteria. However, when the human body is excessively exposed to such a wavelength, the wavelength may cause burns, skin cancer, and cataracts. For this reason, when UV rays are used for sterilization, a user should be careful not to expose his or her skin or eyes to the UV rays and should also be careful about ventilation.

When a UV sterilizer is installed in a vehicle interior for sterilization, it is desirable that the UV sterilizer not be installed in a state of being exposed to the outside of the vehicle. Thus, the above-described problem may be prevented in advance and negatively impacting or degrading the interior design of the vehicle may be avoided.

To this end, as shown in FIG. 1, an UV sterilizer for a vehicle according to an embodiment includes a main body part 100, a sterilization part 200, a driving part 300, and a controller 400.

The main body part 100 is mounted in the vehicle and has a storage area A defined therein.

In an embodiment, the main body part 100 has a rectangular box shape. Further, the storage area A may be configured to have the same box shape in the main body part 100. Here, a lower portion of the main body part 100 is open.

In an embodiment, as shown in FIG. 6, the main body part 100 is mounted adjacent to an overhead console C provided in the vehicle. As shown in FIG. 7, sterilization may be performed in the first row seat when the sterilization part 200 is deployed from the storage area A. This is only one embodiment, and the present disclosure is not limited thereto. The main body part 100 may be mounted at various locations in the vehicle in a state of housing the sterilization part 200 therein. In this manner, sterilization may be performed in the second row seat or the third row seat depending on a vehicle structure.

In addition, the main body part 100 has an open lower portion to be shielded by a cover member 110 on which the sterilization part 200 is mounted. Accordingly, the cover member 110 is exposed to the outside in a state of being horizontal with respect to the roof in the vehicle (refer to FIG. 6). The cover member 110 may be rotated about the lower portion of the main body part 100 in order to withdraw the sterilization part 200 (refer to FIGS. 5 and 7) or deploy the sterilization part 200 refer to FIGS. 4 and 6).

As shown in FIGS. 2A and 2B, the cover member 110 includes a U-shaped connection member 112 connected to the driving part 300 through a rotation shaft 112a. Through this configuration, as shown in FIG. 5, axial rotation of the connection member 112 may allow the sterilization part 200 mounted on the cover member 110 to be selectively withdrawn into or deployed from the storage area A.

The sterilization part 200 is withdrawn into and stored in the storage area A of the main body part 100. When the sterilization part 200 is deployed from the storage area A, the same is configured to selectively emit UV rays to the vehicle interior.

As shown in FIGS. 3A and 3B, the sterilization part 200 includes a mounting frame 210, a heat sink 220, and an ultraviolet-C (UVC) light emitting diode (LED) 230.

The mounting frame 210 is mounted on an upper portion of the cover member 110.

Additionally, the heat sink 220 is coupled to the mounting frame 210 and is formed to be cooled by air flow generated by driving of a cooling fan 222.

In addition, the UVC LED 230 is mounted on the mounting frame 210 in a state of facing the heat sink 220 to dissipate heat to control a temperature rise due to heat generation of the UVC LED 230. Here, a plurality of the UVC LEDs 230 are provided and arranged to emit UV rays at a plurality of locations.

In an embodiment, the UVC LEDs 230 are disposed on a mounting guide 212 at intervals. A first surface 212a and a second surface 212b of the mounting guide 212 respectively have inclinations corresponding to each other with respect to the center of the mounting guide 212. Here, the first surface 212a and the second surface 212b are disposed to have an inclination of approximately 90 degrees. In this manner, when the sterilization part 200 is deployed from the storage area A, UV rays may be emitted to the vehicle interior in a wide range.

In an embodiment, the sterilization part 200 may further include a reflector 240 mounted thereon to surround each of the UVC LEDs 230 and configured to further expand an emission range of the UV rays. Thus, in one embodiment, the sterilization part 200 may include one or multiple UVC LEDs 230 and one or multiple reflectors 240. One reflector 240 may be provided for multiple UVC LEDs 230 or one reflector 240 may be provided for each UVC LED 230 as well.

Accordingly, in the sterilization part 200, the first surface 212a and the second surface 212b of the mounting guide 212 have inclinations respectively corresponding to each other. A plurality of UVC LEDs 230 are mounted on each of the first surface 212a and the second surface 212b, thereby reliably emitting UV rays to the center of the first row seat and the opposite sides of the vehicle interior. Additionally, the emission range of the UV rays may be further expanded by the reflectors 240.

Here, the reflectors 240 may have various and/or complex shapes such as a convex shape, a concave shape, and/or a wavy shape in order to expand the emission range of UV rays.

Meanwhile, the driving part 300 is coupled to the main body part 100 and is formed to provide rotational driving force to allow the sterilization part 200 to be withdrawn into or deployed from the storage area A.

The driving part 300 includes a housing 310, a driving motor 320, a gear member 330, a clutch gear 340, a shaft member 350, and a clutch plate 360.

First, the housing 310 is a case and is coupled to the rear side of the main body part 100.

Additionally, the driving motor 320 is coupled to the inside of the housing 310 and provides rotational driving force in response to an input control signal.

The gear member 330 is connected to the driving motor 320 and is formed to transmit rotational driving force provided from the driving motor 320 to the clutch gear 340.

The gear member 330 may include a first worm gear 332, a worm wheel gear 334, and a second worm gear 336, as shown in FIG. 9, in order to transmit rotational driving force.

Accordingly, rotational driving force of the driving motor 320 is transmitted to the first worm gear 332, the rotational driving force transmitted to the first worm gear 332 is transmitted to the worm wheel gear 334, and the rotational driving force transmitted to the worm wheel gear 334 is transmitted to the second worm gear 336 in a sequential manner. The clutch gear 340 engaged with the second worm gear 336 is thereby selectively rotated.

The clutch gear 340 is engaged with the second worm gear 336 to be rotated in conjunction with the second worm gear 336, thereby selectively rotating the clutch plate 360.

The shaft member 350 is connected to the connection member 112 provided in the sterilization part 200 through the rotation shaft 112a (refer to FIG. 2B). Accordingly, when the shaft member 350 is rotated, the connection member 112 is axially rotated. Through this configuration, the cover member 110 is rotated relative to the main body part 100. As such, the sterilization part 200 is withdrawn into or deployed from the storage area A.

The clutch plate 360 has a protrusion member 362 formed thereon and located in a locking groove H provided on the outer peripheral surface of the shaft member 350. In this manner, the clutch plate 360 is rotated in conjunction with the shaft member 350.

In addition, the clutch plate 360 is disposed to make surface contact with the inner peripheral surface of the clutch gear 340. Here, when the clutch gear 340 is rotated through rotational driving force transmitted from the second worm gear 336, the clutch plate 360 is rotated in conjunction with the clutch gear 340 to rotate the shaft member 350.

In other words, when rotational driving force provided from the driving motor 320 is transmitted as shown in the arrow direction depicted in FIGS. 8D and 9, elastic force is provided in a state in which the position of an elastic member 380 is fixed by a stopper member 370. Accordingly, the clutch plate 360 may be locked and fixed in the locking groove H.

In this state, when the clutch gear 340 is rotated by rotational driving force transmitted from the second worm gear 336, the clutch plate 360 is in surface contact with the inner peripheral surface of the clutch gear 340 and is also rotated by elastic force. At this time, when the clutch plate 360 is rotated, the shaft member 350 is also rotated through the protrusion member 362 locked in the locking groove H.

As a result, as shown in FIG. 8A, the rotation shaft 112a is rotated by rotation of the shaft member 350 of the driving part 300 and rotational driving force of the rotation shaft 112a is transmitted to the connection member 112. As shown in FIGS. 8B and 8C, the cover member 110 having the sterilization part 200 mounted thereon is rotated by rotation of the connection member 112. The sterilization part 200 is thereby permitted to be deployed from the storage area A and positioned toward the vehicle interior.

Meanwhile, upon determining that the storage area A is open as described above, the controller 400 controls the operation of the sterilization part 200 for emission of UV rays. At this time, after UV rays are emitted for a predetermined time, the driving motor 320 is controlled to rotate the shaft member 350 in the reverse direction. In this case, if it is determined that a driving interruption event has occurred, rotational driving force may not be transmitted by the controller 400 in a state in which the sterilization part 200 is deployed. At this time, the cover member 110 is rotated to an initial position thereof through manual operation.

In other words, if an event such as discharge or driving motor failure occurs, it is not possible to provide rotational driving force to allow the sterilization part 200 to be withdrawn into and stored in the storage area A. For this reason, a user may then manually press or push the cover member 110 so that the deployed sterilization part 200 may be returned to the withdrawn state and stored in the storage area A.

Here, when manual operation is performed to press the cover member 110 without controlling the controller 400, configured to otherwise perform transmission of rotational driving force, the connection member 112 is rotated and the rotation shaft 112a is also rotated. Here, the shaft member 350 is also continuously rotated.

In this case, as shown in the arrow direction depicted in FIG. 11, rotational driving force transmitted from the shaft member 350 rotates the clutch plate 360 by the protrusion member 362 locked in the locking groove H. Here, rotational driving force transmitted to the clutch plate 360 is not transmitted to the clutch gear 340 and rotational friction is generated against the clutch gear 340. Consequently, the clutch gear 340 is maintained in a fixed state.

As shown in FIG. 10A, due to independence of the clutch gear 340 and the second worm gear 336, transmission of rotational driving force is not performed during reverse rotation, as shown in the arrow direction depicted in FIG. 10B. The clutch gear 340 is also not rotated by the clutch plate 360 and is in a fixed state by rotational friction (refer to FIG. 11). As a result, when an event occurs, the cover member 110 may be manually and forcibly rotated in a state in which the sterilization part 200 is withdrawn from the storage area A, thereby enabling the sterilization part 200 to be withdrawn into and stored in the storage area A.

Therefore, in the present embodiment, the controller 400 performs a control operation to transmit rotational driving force in the forward and reverse directions of the driving part 300. This enables the sterilization part 200 to be stored in or deployed from the main body part 100. In addition, in the event of an event such as discharge or driving motor failure, manual operation is also optionally performed. This prevents the sterilization part 200 from being exposed to the outside when the event occurs in the deployed state of the sterilization part 200.

The present disclosure uses UV rays to sterilize a vehicle interior An UV sterilizer of the present disclosure selectively switches from a storage state to an operation or deployed state for sterilization. This has an effect of preventing a safety problem and avoiding negatively impacting interior design of a vehicle due to the UV sterilizer installed in an exposed state.

In addition, the present disclosure has a plurality of UVC LEDs mounted on a mounting guide having inclinations corresponding to and complimenting each other. Also, a reflector is mounted on each UVC LED. These features have an effect of expanding a sterilization area in the vehicle when the UV sterilizer switches to the operation state for sterilization.

The present disclosure has been described in detail with reference to various embodiments shown in the drawings, but the embodiments are merely illustrative. It should be appreciated by those of ordinary skill in the art that various modifications may be made from the embodiments, and all or a part of the embodiments may be selectively combined with each other. Therefore, the true technical protection scope of the present disclosure should be defined by the technical spirit of the appended claims.

What is claimed is:

1. An ultraviolet (UV) sterilizer for a vehicle, the UV sterilizer comprising:
a main body part mounted in the vehicle, the main body part having a storage area formed therein;
a sterilization part configured to be withdrawn into and stored in the storage area and to be deployed from the storage area to selectively emit ultraviolet rays to a vehicle interior;
a driving part coupled to the main body part and configured to provide a rotational driving force to deploy the sterilization part from or to withdraw the sterilization part into the storage area; and
a controller configured to selectively control operations of the main body part, the sterilization part, and the driving part.

2. The UV sterilizer of claim 1, wherein the main body part includes a cover member configured to shield an open interior of the main body part in the vehicle interior and wherein the sterilization part is mounted on the cover member.

3. The UV sterilizer of claim 2, wherein the cover member comprises a U-shaped connection member connected to the driving part through a rotation shaft.

4. The UV sterilizer of claim 3, wherein the connection member is configured to rotate to allow the sterilization part to be selectively withdrawn into and deployed from the storage area.

5. The UV sterilizer of claim 2, wherein the sterilization part comprises:
a mounting frame mounted on the cover member;
a heat sink coupled to the mounting frame and configured to be cooled by an air flow generated by a cooling fan; and
a plurality of UVC LEDs mounted on the mounting frame in a state of facing the heat sink and formed to emit the ultraviolet rays at a plurality of locations.

6. The UV sterilizer of claim 5, wherein the sterilization part further comprises a reflector mounted on each UVC LED of the plurality of UVC LEDs and configured to expand an emission range of the ultraviolet rays.

7. The UV sterilizer of claim 5, wherein the plurality of UVC LEDs are disposed at intervals on a first surface and a second surface of the mounting guide, and wherein the first surface and the second surface have inclinations respectively corresponding to each other with respect to a central portion of the mounting guide.

8. The UV sterilizer of claim 1, wherein the driving part comprises:
a housing coupled to a rear side of the main body part;
a driving motor coupled to an inside of the housing and configured to provide the rotational driving force;
a gear member connected to the driving motor and configured to transmit the rotational driving force generated from the driving motor in response to an input control signal;
a clutch gear engaged with the gear member to be rotated in conjunction with the gear member;
a shaft member connected to a connection member provided in the sterilization part through a rotation shaft; and
a clutch plate locked in a locking groove provided in the shaft member to be rotated in conjunction with the shaft member.

9. The UV sterilizer of claim 8, wherein the clutch plate is disposed to make surface contact with an inner peripheral surface of the clutch gear and is rotated in conjunction with the clutch gear when rotated by the rotational driving force transmitted from the gear member, thereby rotating the shaft member.

10. The UV sterilizer of claim 8, wherein the clutch plate is rotated in conjunction with rotation of the shaft member when the sterilization part is manually stored in the storage area and generates rotational friction in a state of making surface contact with an inner peripheral surface of the clutch gear.

11. The UV sterilizer of claim 8, wherein the driving part further comprises an elastic member mounted on an outer peripheral surface of the shaft member and configured to allow a stopper member to fix a position thereof, and wherein the elastic member provides elastic force to the clutch plate to allow the clutch plate to be fixed in the locking groove.

12. The UV sterilizer of claim 1, wherein the controller is configured to provide the rotational driving force to the driving part to deploy the sterilization part and open the storage area and to control, upon determining that the storage area is open, the operation of the sterilization part for emission of the ultraviolet rays.

13. The UV sterilizer of claim 12, wherein the controller is configured to provide, upon determining that a predetermined sterilization time has elapsed, reverse rotational driving force to the driving part so that the sterilization part is rotated and is withdrawn into and stored in the storage area.
